(19) United States
(12) Patent Application Publication
(10) Pub. No.: US 2024/0119422 A1
(43) Pub. Date: Apr. 11, 2024

(54) Work Profile Alignment

(71) Applicant: Almas Insight, Inc., Seattle, WA (US)

(72) Inventors: Brayden W. Olson, Bellevue, WA (US); Robert A. Savette, Seattle, WA (US)

(21) Appl. No.: 18/379,122

(22) Filed: Oct. 11, 2023

Related U.S. Application Data
(60) Provisional application No. 63/415,238, filed on Oct. 11, 2022.

(51) Int. Cl. G06Q 10/1053; A61B 5/00; G06V 40/16

(52) U.S. Cl. CPC G06Q 10/1053; A61B 5/0006; G06V 40/174

(57) ABSTRACT

Technology is described for determining a candidate's suitability for a work profile. The method can include identifying a candidate's abilities by obtaining the candidate's electronic response to a plurality of problems presented in a work simulation. Another operation may be capturing a candidate EEG (Electroencephalogram) during an electronic response provided by the candidate. A normal neuro-mappings for the plurality of problems may be obtained based in part on aggregated EEG data for a group of individuals for the plurality of problems solved in the work simulation. The candidate EEGs for the plurality of problems can be compared to the normal neuro-mappings for the plurality of problems. Candidates may be scored based in part on the amount candidate EEGs diverge from the normal neuro-mappings.

### PRIORITY DATA

This application claims the benefit of U.S. Provisional Patent Application Ser. No. 63/415,238, filed Oct. 11, 2022, which is incorporated herein by reference.

### BACKGROUND

For almost a century, behavioral scientists have labored to develop precise mechanisms to quantify, characterize, and represent human capabilities (also known as soft skills) in a pseudo-language that would create the ability to predict probable results for use in the real world.

There are many types of non-technical skills or “soft” skills that employers are looking for in employees, and such non-technical may be more difficult for employees to learn than technical skills. These skills may include human skills, cultural traits, competencies, communication skills, leadership skills, decision making skills, etc. Whatever employers call these non-technical skills, 97% of surveyed employers agree that such non-technical skills are as important or more important than technical skills. Employers further acknowledge that 89% of new hire failures are with candidates who are lacking in the important “soft” skills.

The business impact of understanding the soft skills that candidates or potential employees may offer to employers can be significant. In a first example, a company may be hiring their own talent. A company may need the right person in the right project at the right time. By fundamentally understanding potential new hires and even current employees, a company can assemble a talent base built on desired skills and capabilities. Such insight may save tens of millions in contractor's fees and improve performance outcomes with employee hires or by avoiding underperforming employees.

Another business impact of understanding the skills of potential hires and current employees is the ability to reduce attrition. Certain individuals fit better in certain roles, projects and cultures. Especially in high sensitivity roles (e.g., executives), it can be hard to find the right individuals to fit certain roles unless the organization is able to identify the attributes of a person to fit a desired work profile. When these talent acquisition processes are successful, the processes may even save the organization millions of dollars.

### BRIEF DESCRIPTION OF THE DRAWINGS

FIG. 1 is a block diagram illustrating an example of a system for determining a candidate's suitability for a work profile.

FIG. 2 is an example of EEG-assisted capability measurement operations for candidates.

FIG. 3 is a block diagram illustrating an example system for comparing a candidate's capability profile to a work profile or role using a work simulation application and related systems.

FIG. 4 is a flow chart illustrating an example of collecting data for an insight index and a differential comparison that may be performed.

FIG. 5A illustrates example charts for operations of comparing geomaps.

FIG. 5B is flowchart illustrating an example of operations for comparing a candidate's ability profile to a work profile.

FIG. 6 is a chart illustrating an example of different types of comparisons or matching which may include team matching.

FIG. 7 illustrates an example listing of candidate or employee capabilities.

FIG. 8A illustrates an example of a graph of connection and/or influence identified for an individual within an organization.

FIG. 8B illustrates a flowchart for a method for determining a candidate's suitability for a work profile.

FIG. 8C is a flowchart illustrating an example of a method for comparing a candidate's capability profile to a work profile.

FIG. 9 is a block diagram illustrating an example of a service provider environment (e.g., a public or private cloud) upon which the comparison instrument or other processes for this technology may execute.

FIG. 10 is a block diagram illustrating an example of computer hardware upon which this technology may execute.

### DETAILED DESCRIPTION

Reference will now be made to the examples illustrated in the drawings, and specific language will be used herein to describe the same. It will nevertheless be understood that no limitation of the scope of the technology is thereby intended. Alterations and further modifications of the features illustrated herein, and additional applications of the examples as illustrated herein, which would occur to one skilled in the relevant art and having possession of this disclosure, are to be considered within the scope of the description.

This technology may utilize artificial intelligence, analytics and other techniques that enable the user to convert human capabilities to multi-dimensional representations. Those multi-dimensional representations can be used in a pseudo-language to deliver actionable business analysis (talent acquisition, employee attrition, work group creation, etc.) An example of one of these representations is a Euclidian geometric representation of individuals' or groups' work capabilities.

The present technology also obtains a digital work sample from potential employees or existing employees by providing an electronic environment which may contain: avatars, visual context, video context, audio context, financial data, questions, scenarios, and work problems which simulate making decisions that reflect choices replicating real world conditions. Obtaining this digital work sample may also use simulation methods that may include: simulated client personnel, simulated office spaces, simulated supervisors, branding and other work simulation elements.

An electronic work simulation or work simulator may include the opportunity for the candidate or employee to: respond to situations, make choices, provide responses and answer problems or answer questions. Examples of choice models that may be presented to individuals or users in the electronic work simulation or work simulator may include: divergent path, multiple choice, multi-select, sliding scale, open answer, peer impacted (multiplayer) and peer-reviewed open answer choices. These choices are neither inferred nor self-reported, but the individual's answers are demonstrated in the context and problems presented by the overall simulation. Differing testing methods may be tied to specific capabilities for candidates across many capabilities.

In one configuration of the technology, neuro measurement devices may provide sensor readings captured from individuals or users, and the sensor readings can be used with digital work samples to identify benchmark neurological patterns for good performance in selected capabilities. In contrast, neurodivergent behavior can also be identified. As an example, the technology may identify electromagnetic patterns in highly analytic individuals when performing well or performing at a desired level while responding to cognitive questions. On the other hand, neurological measurements may be identified for neuro-divergent individuals or low performance when answering visual and/or emotional questions by comparing incoming neurological measurements using the normal (i.e., a defined group of neurological measurements or an average from a selected group) or pre-recorded electromagnetic patterns as a baseline.

More specifically the neuro-measurements in the technology may be used for detection of specific types of problematic psychological issues. For example, neuro-measurements may be used to analyze lying, sociopathy or psychopathy (e.g., dark triad traits). In measuring sociopathy, for example, a user may be presented with an emotionally charged video of an office situation with one of their co-workers beginning to cry, and the user may be asked to decide about how to respond between N number of response choices. As the candidate watches the video and responds, brain activity may be captured. The normal level of brain activity and region of response in the brain to the video can be used as the baseline for the response. Neurodivergent behavior can then recorded from some candidates and those with limited neuro activity or inappropriate neuro response (e.g., response in the wrong portion of the brain) may be correlated to sociopathic behaviors.

In another configuration, facial expression recognition may be used to detect incorrect emotional or mental states. For instance, the process may be able to detect confusion in a facial expression of a candidate when the candidate is presented with a question. The facial expressions of the candidate may be captured through cameras (e.g., visual, infrared or other types of cameras). Then the facial expression can be tested to see whether the candidate or user is being honest or not. The facial recognition can also test candidates to see if the person is emotionally reacting in a way that is expected based on the question presented in the work simulation application.

Data from the situational responses of the work simulation application can be correlated with the neurodivergent mapping or visual cues from facial expression mapping to determine whether the candidate has neurodivergent behavior or the candidate is lying or confused. More specifically, the EEG readings or camera facial expression readings can be correlated to the candidate's response in the work simulation application. This correlation may help the system determine if a candidate is a sociopath. Similarly, the correlation may assist with determining the emotions a person is feeling. For example, is the person sad, happy or confused when a specific question is being answered and is that an appropriate feeling when addressing that problem or question.

As mentioned, normal EEGs can be compared to the EEGs of a person who has provided responses in the work simulation application. This may result in detecting a difference between the two EEGs and detecting whether there is a neurodivergent response. In one example, a person lying can be seen on the EEG as compared to a normal EEG for a specific scenario or question. This is because the person has to think through a socialized response to lie and then the candidate gives a different response than their initial mental reaction. What the EEG detects can also be compared to the actual recorded response for a problem or question in the work simulation application. The person may have responded to a question that the person was happy but actually the EEG says the person did not really know what the emotional state for the question was and the person is making up a response. In another example, the EEG may detect that the person being tested does not understand how a person presented in the simulation scenario was feeling. Such results can be provided by correlating 1) the EEGs of candidates to normal EEGs or a grouping of EEGs and 2) correlating the EEG with the response in the work simulation application (e.g., a typed in response or selected response). When items do not correlate correctly, a determination can be made that a candidate is likely displaying psychopathic behavior or the person being dishonest or lying. For example, a back portion of the human brain handles emotions and that part of the brain should have detected activity or “light up” for certain kinds of questions. But the front part of the brain is activated when we lie or generate a response (socially deriving an answer). So, when the wrong brain pattern is found to be activated but the candidate says they understand the question and response, the correlation will prove this assertion by the candidate to be wrong.

In a related example, facial recognition can detect facial responses using a common set of questions. The results of the facial recognition can detect a candidate's certainty or confidence as opposed to confusion about questions or scenarios in the work simulation application. Confusion in the face of a candidate can be scored lower than certainty or confidence when a question is answered.

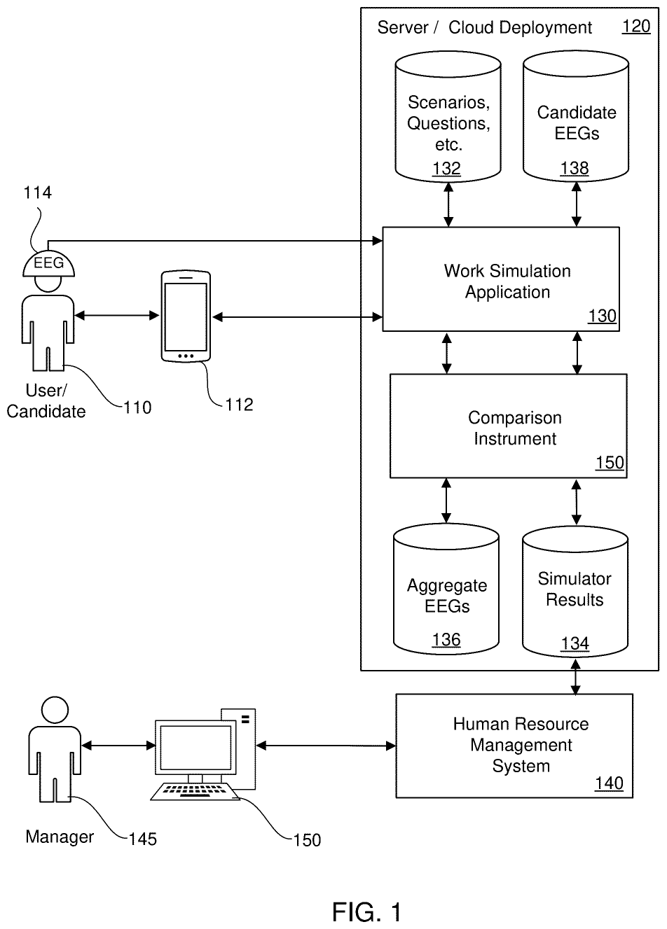

FIG. 1 illustrates a block diagram of a system for determining a candidate's suitability for a work profile or suitability for a role. A candidate's 110 abilities or user abilities may be tested by obtaining the candidate's electronic responses to a plurality of problems, questions or scenarios 132 presented in a work simulation application 130. The work simulation application 130 may be located on a server or in a service provider environment 120 such as a cloud environment (e.g., Amazon AWS, Microsoft Azure or Google cloud).

A candidate's EEG (Electroencephalogram) 114 may be captured during a response to questions or scenarios 132 provided by the candidate to the work simulation application 130. The candidate EEGs may be stored in a candidate EEG data store 138. Normal neuro-mappings 136 (or common neuro-mappings) for the plurality of problems can also be obtained. The term normal in the context of the neuro-mappings may represent a medical normal neuro-mapping, an estimated normal neuro-mapping, a sampled normal neuro-mapping from one specific population of individuals or a group of samples where a common, mean, average, minimum, maximum, or clustering of neuro-mapping. Accordingly, groupings of EEG readings may be summarized using statistical or other mathematical functions to provide metrics that are usable for comparison. Aggregated EEG data of normal neuro-mappings 136 for a group of individuals for a plurality of problems solved in the work simulation may be processed and stored in a data store as baseline or aggregate EEGs 136 to provide the normal neuro-mappings when solving certain types of problems.

A comparison instrument 150 can be a service provided to receive information from the work simulation application 130 and to compare candidate EEGs for solving problems to the normal neuro-mappings 136 for the same problems being solved. Then the comparison instrument 130 can score candidates based in part on the amount candidates' EEG measurements diverge from the normal neuro-mappings. These scores may be stored in the simulator results data store 134. A human resource management system 140 may have access to the scores or the human resource management systems 140 may be able to retrieve the simulator results and perform further calculations or transformations on the scores. The human resource management system 140 may be accessed by a manager 145 through a computing system 150 (e.g., desktop, mobile device, tablet, etc.).

In one example, the raw scoring of answers or feedback from the work simulation application may be adjusted based on detected dishonesty or uncertainty detected by the comparison instrument 150, the work simulation application 130 and/or the human resource management system 140 using the EEG measurements as compared to normal EEGs or the EEG measurements as correlated to responses to the scenarios, questions, or problems presented in the work simulation application 130. In another example, the scoring may be adjusted based on the candidate answering questions with emotional material where a determination is made that sociopathy has been identified.

The work simulation application 130 can remove interview bias. High extroversion or agreeable personality types that might normally ‘game’ traditional measurements or provide false positives in an interview and ace interviews have no significant advantage in a work simulation application 130.

This technology can provide an advantage in finding talent for hiring. This system and method can find talent that traditional, self-reporting methods may miss. Prior methods show socio/economic biases toward those with certain education and employment status but the work simulation application 130 generally has less of such biases.

The work simulation application is generally straight forward to use and is engaging for users to work with. Users heavily preferred being evaluated in a work simulation over other types of surveys, test, interviews or measurement methods for evaluating soft skills. Users also reported the method provided a clear, complete understanding of their choices. Analysis has shown that the simulations provide an understanding of the user's choices that are two times clearer than written cases and four times clearer than 180 surveys (e.g., employees feedback on managers).

These systems and methods can be descriptive and predictive. Users report that this method was by far the most accurate assessment of their performance compared to traditional methods. The data produced by this system and method provided data highly correlated to manager reported performance.

The work simulation can also be used to identify baseline measurements for employees who churn quickly and those who last longer in their type of organization. This allows the organization to better hire, onboard and retain talent. Any baseline data created by the system is created based on comparison instruments or methods that have not demonstrated adverse impacts in areas like gender, race bias or other protected demographics.

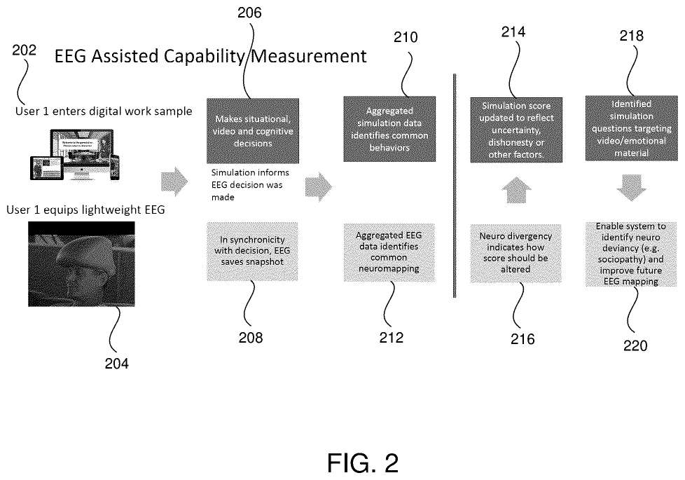

FIG. 2 illustrates an example of EEG-assisted capability measurement operations. This collection method can provide an insight index where a normal EEG can be compared with a subject's input EEG and this may result in a differential scoring. One part of the indexing is that the user may enter a digital work sample 202 using the work simulation application. In addition, the user may equip a lightweight EEG 204 during the use of the work simulation application.

The user or candidate may make decisions in response to situational, video, audio and business presentations 206. The work simulation application may be able to receive an EEG reading because an EEG detectable decision is being made. When an EEG detectable decision is made, the EEG reader device can take a snapshot of the user's brain wave state 208 during the decision window. For example, a time window may be defined for a period of time before, during and/or after the decision is being made and an EEG may be captured during that time window.

Aggregated simulation data can be used to identify common behaviors 210 and actions during the work simulation. Aggregated EEG data can be used to identify normal EEG patterns 212 or neuro-mappings during the decision-making operations for the common behaviors and actions in the work simulation. A comparison of the user's EEG pattern(s) can be made to the normal EEG patterns captured during the simulation behaviors.

The scores from the work simulation application can be modified based on the amount of neuro-divergency 216 detected between the user's EEG patterns and the normal EEG patterns. The user's scores in the simulation can be updated 214 to reflect uncertainty, dishonesty, sociopathy or other factors detectable with the comparison of the EEG patterns. The scoring system can also identify simulations or simulation questions targeting video or emotional material 218. These questions can be used to identify neuro-deviancy 220 (e.g., sociopathy, psychopathy, etc.) and improve future EEG mappings.

The data output from this system can also help improve the work simulation. The work simulation learns what uncertainty, dishonesty and other neuro-divergency looks like on the EEG and then an incoming EEG with those attributes can automatically modify those scores as the incoming EEG is used for training and becomes part of the data store. Similarly, the work simulation provides a context (e.g., a video, reading passage, animation) to trigger certain normal emotional or behavioral responses and enables the EEG to identify emotional or behavioral responses such as sociopathy which are not normal.

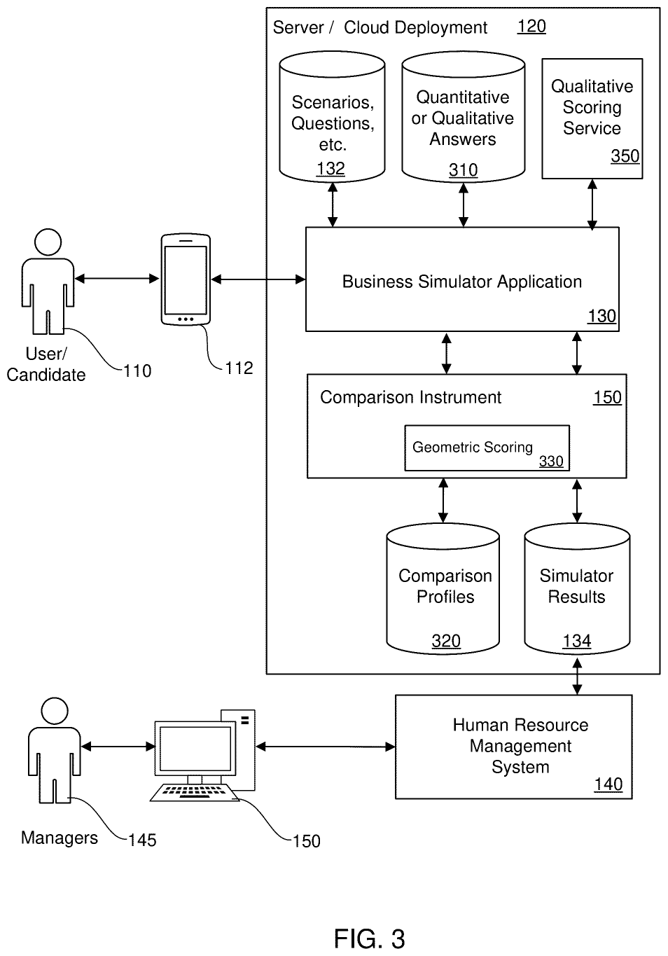

FIG. 3 illustrates a system for comparing a candidate's capability profile to a work profile or role using a work simulation application 130 and related processing systems. In this configuration, the user 110 may provide quantitative answers 310 which can be scored and stored using a data store for quantitative answers 310. The system may also have qualitative answers which can be used to train machine learning systems and the machine learning may grade the input from the user 110, accordingly.

The technology may include a comparison instrument 150 that is a service located on a server 120 or cloud deployment. This comparison instrument 150 may receive input from the work simulation application 130 and inputs from a data store containing comparison profiles 320 from high performing employees of an organization or similar benchmarks. The output of the comparison instrument 150 may be a value that represents a probability, a scalar value, an alpha numeric grading or a percentage representing whether a candidate matches with a role or work profile. The comparison instrument 150 can receive the inputs and transform them into comparison instrument output that is stored as simulator results 134 representing a candidate match to a work profile or role.

The machine scoring may also be role specific and organization specific. The user data sets may vary but there may be data from a provider of the work simulation application to help fill in work profiles where needed. The parameters a human administrator (e.g., HR manager) sets may be defining top performing candidates to be used as template or benchmark for specific roles. Then the system identifies the statistically significant differences between the top performers and others (e.g., other employees in the full data set of profiles).

The work simulation application 130 may include problems or scenarios that are peer impacted. In a first example, Player 1 may decide to allocate budget to employee development programs. Player 2 may then see three options, two of the options may relate to/are strengthened by employee development programs with one option highly leveraging this budget and a third option which is totally independent. Such a question may measure collaboration between players and see if Player 2 is willing to collaborate with Player 1. In a second example, Players 1, 2 & 3 decide between four potential projects that would impact company performance by vote. If the four players vote on potential project 1, then players 3, 4 & 5 can receive decisions exclusive to problems where potential project 1 is selected.

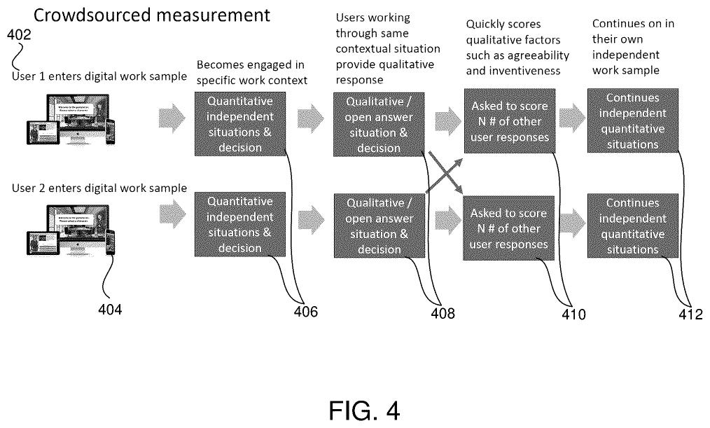

FIG. 4 illustrates one example of a method for collecting data for an insight index and a differential comparison that may be performed. A first user (e.g., User 1) may provide a first digital work sample 402. A second user (e.g., User 2) may provide a second digital work sample 404. Both users can be engaged in the specific work context provided in the work simulation application. The users may be asked to work in independent situations and make independent decisions that can be quantitatively measured 406.

The users may then be presented with the same context situation and may also be asked to provide qualitative responses 408 to certain types of questions for the situation. These qualitative answers may be considered open answers for the situation and/or decisions to be made. The qualitative answers may be written answers, video answers, audio answers or another type of answer which may be obtained from the user or candidate through the work simulation application.

In one configuration, the users or candidates may be asked to score one or more responses of other users 410. The peer users may grade the qualitative response on criteria such as inventiveness, agreeability, reasonableness, logic, possible outcomes, cost and similar criteria. The users may then be asked to continue working in their own independent work samples. The continued questions in the work simulation application may be quantitative questions 412 or situations that can be scored quantitatively by the work simulation application.

In a more specific example, the questions in the work simulation application may be peer reviewed. For instance, in measuring creativity, a common business problem can be presented. This problem may be regarding best strategies to handle the shift to remote work environments while maximizing performance and this question may be answered by Player 1 in an open answer format. The other players in the simulation may be asked to provide scoring using a thumbs up/thumbs down, 1-100 scalar score, or a highly agree to highly disagree score with N other player's responses both in terms of whether they AGREE with the solution and whether they find the solution INNOVATIVE/CREATIVE. Other players who play after Player 1 then see Player 1's response and rate the response in real time. This may also go a layer deeper where other players rate the reviews of Player is answer. If a review of a response is very low and outside the player's response cluster, then that review may be thrown out.

Once N number of users have rated Player is response, this aggregated score may be compared to averages for that question in the system. Those within 1 standard deviation are scored as a 2 (or equivalent score). Those more than 1 SD below are a 1 (or equivalent) and those above 1 SD (or equivalent) are a 3. The scores for both agreeableness and innovation may be scored as both independent and combined capability factors.

In these configurations, the candidates can be put in the same simulation situation where they may solve a problem and then they will perform an evaluation of peers who were in the same simulation or same problem. Then N other candidates rate a solution as to how good or agreeable the solution is and how inventive the solution is. When enough candidates have provided an evaluation, then this provides a qualitative score for the answer. This is type of crowd scoring but each of the scorers have been through the simulation and addressed the problem themselves. This assists with scoring a qualitative area such as inventiveness, which is otherwise difficult to score quantitively. Certain qualitative problems or scenarios can produce more accurate results by using this scoring method rather than quantitative scoring methods.

Sentiment analysis, is another type of qualitative analysis that may be built over time using machine learning. Candidates may score the sentiment of other candidates with the simulation. Over time, a system that is using natural language processing, text analysis, and/or computational linguistics can be trained to determine what someone's response means, as the candidates provide training examples to the sentiment system. Thus, the system may measure personality, capability, or leadership. The comparison instrument can score what the candidates are seeing, and can look the commonality in the sentiment in the answers. Then the system can score the qualitative answer(s). Sentiment may be scored by the qualitative scoring service too using artificial intelligence.

The present technology can measure human behavior. Actual behavior of individuals is a common language for every work place. The work simulation application can collect information on multiple work relevant behaviors that employers care about underneath each capability and report about the work relevant behaviors for candidates or employees. Depending on the role and context, some capabilities may be desired that could be considered detrimental in other roles. Whether an individual's capabilities fit a specific work profile is not a right or wrong answer but a question of determining a fit for a role or position.

In a limited time (e.g., under a few hours), an organization can begin to see objectively demonstrated capability data for a candidate providing insight that can solve a variety of business issues. The data may then be seamlessly integrated into whatever talent management product is desired. The acquired data immediately replaces “self-report” data or no data at all with actionable business data.

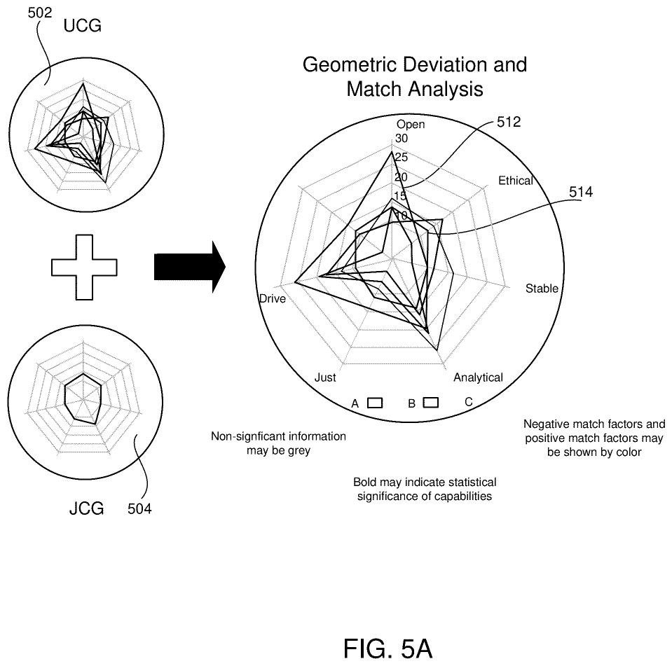

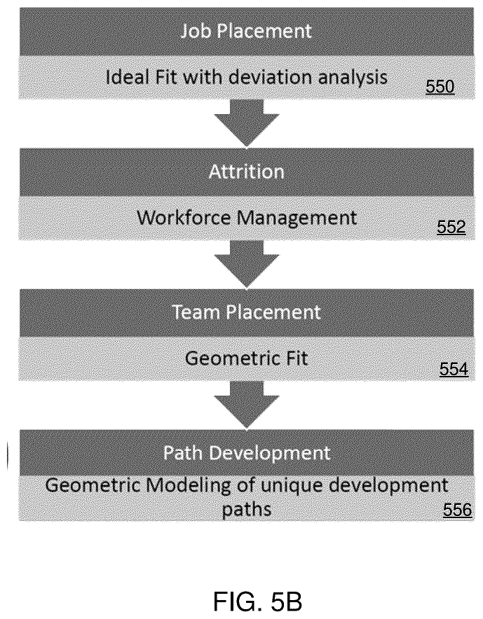

FIG. 5A illustrates example charts for operations of comparing a unique capability geomap (UCG) 502 (or a candidate's capability profile) to a job capability geomap (JCG) 504 (e.g., work profile or capability profile) using a comparison instrument. However, the comparison instrument may include an additional geometric scoring module 350 (FIG. 3). The geometric scoring module 350 can obtain a quantitative data set representing a quantitative scoring of a candidate's abilities based on the candidate's responses to problems or questions presented in the work simulation application. For example, a score may have been generated for various capabilities and behaviors that the work simulation application is evaluating or asking for action on.

A qualitative data set may be generated that represents a qualitative scoring of the candidate's abilities as scored by a qualitative scoring service 350 (FIG. 3). The quantitative data set and the qualitative data set 310 can be mapped into a space with two, three, four or more dimensions to form a unique capabilities geometric object 512, as in FIG. 5. The unique capabilities geometric object 512 (e.g., capabilities index) for the user(s) or candidate(s) can be compared to an ideal geometric object 514 representing desired capabilities of an ideal candidate. The quantitative answers of a candidate can be scored by the work simulation application and may then be entered into the candidate's profile. The term geometric object is also defined here as data modeled in N dimensions and hyper dimensional comparison for the data and/or vectors may be used. In 2D (two-dimensional) form, the unique capabilities geometric object 512 may be a polygon on a polygon chart or circle chart with vertexes the represent an individual capability or a group capability and its magnitude. In 3D (three dimensional) form, the unique capabilities object may be a 3D object and the vertexes of the object may be individual or group capabilities and magnitudes. Similarly, the ideal geometric object 514 may have vertexes that represent desired capabilities and numerical magnitudes.

A geometric deviation and match analysis can be performed in the comparison. The analysis can determine whether the unique capabilities geometric object 512 (e.g., input object) deviates from the ideal geometric object in a plurality of capability areas in a positive, negative or neural amount. In another example, the areas or volumes of the unique capabilities geometric object and ideal geometric objects may be compared. Further, a match value may be computed using the Euclidean distance of the capability values from the desired role capability values. The system may then determine whether or not to recommend the candidate profile based on an aggregation of the amount of deviation measured in multiple capability areas. If the candidate profile does not deviate significantly in a negative amount from the ideal geometric object, then a recommendation may be made to hire or retain the candidate, employee or user. Alternatively, if the candidate, employee or user does deviate in significant amounts in important areas, then a recommendation may be made not to hire or retain the candidate or user.

The ideal geometric object may be created by using the profile(s) of one or more high performers (e.g., an average of a few high performers) and the reference profile may be compared statistically to the input profile or candidate profile. Not all areas of capability may be compared but the areas that are compared can be statistically significant. When top performers for a role are compared to average performers for a role, some capabilities do not matter or are not relevant to the performance of the role or work profile. There may be areas of overlap in the ideal geometric object and the incoming geometric object that are not statistically significant and may not be considered in a comparison.

In addition, the comparison instrument can distinguish whether the compared capability should be above the threshold or below the threshold for a capability. For example, a positive trait that is over the threshold is good because a candidate may have more of that positive trait. By comparison, being under the threshold is good for a negative trait but being over the threshold for a negative trait is not desirable. As a more specific example, you may want a candidate to be low in experimentation if they are an accountant. In some situations, a candidate can be more than a 100% match in relevant capabilities. For instance, someone can score 9 out of 7 (the threshold) and because the candidate is high in areas that are statistically significant to performance in the role, then the candidate can be considered to exceed the standard and be a good fit for the role.

FIG. 5B further illustrates areas where the present technology may be used in a business over time. A deviation analysis may be used where a candidate's capabilities are compared with an ideal capabilities profile for potential job placement, as in block 550. In order to enable job placement, baseline data for roles may be setup. The baseline for a role may include adding (or removing) candidates, employees or members to a group to provide the relevant metric information for the baseline's users. The baseline data for roles may be used for the talent scan or role matching. Groups may also be setup with a name for the group members that may be added to the group.

A talent scan or role matching may display a list of all candidates or members available for matching, along with various pieces of data about them. There may be two modes of fit computation—Auto and Manual. Auto mode estimates an employee's fit for a given role using the baseline data, whereas Manual mode may compute how closely the candidate matches a selected set of capabilities (e.g., by providing numerical or labeled comparison output) so that a user may browse through the candidate matches. The candidates or members of groups may have performance values. When adding these performance values into a baseline or group, the performance values may be modified if needed, and the baseline can then immediately be used to produce consistent results for the role matching or talent scan function.

The technology may provide a talent scan or role match to find candidates in the database that match certain soft skills or filter out a candidate who does not match the desired qualitative skills. A decision tree classifier can be created which is an AI (artificial intelligence) process that is trained to predict which segment (High Performance, Medium Performance, or Low Performance) a candidate falls into based on their capabilities and qualitative attributes of the candidate. The decision tree classifier can be created based on attributes or data known about the members of the selected role baseline and their provided performance level(s). In manual mode, instead of using a decision tree classifier, a sum of the selected capabilities is used to compute the fit value. Summations that are 80% or more of the theoretical maximum are classified as a high fit.

The comparisons may also be used for workforce management to help reduce attrition, as in block 552. This may occur by getting employees better role fits and/or in getting employees better training based on their unique capabilities. In one example, an automatically identified desired candidate profile may combine created behavioral data with objective human capital management data from the client's HCM (Human Capital Management) system. For candidates, role baselines may be combined with attrition insight derived from a baseline data store system. So, this processing can use data generated from two areas combined together. For high potential candidates, time-to-promotion data and employee tenure data from the HCM data may be used. For attrition, employee tenure data and location data can be used from the HCM data. For leadership succession, management reviews (given/received), time-in-role data for reports and a variety of organizational performance metrics (such as sales) may be used.

One example of attrition analysis may be used for candidates. The geometric object may display the average of the user capability values in the attrition segment. Other attrition information may come from analysis of the generated decision tree classifier, which is an AI-driven process that is trained to predict which segment someone is in based on their capabilities. The process shows which capabilities contribute to attrition and by how much.

In one configuration, an attrition analysis for candidates may be performed. A decision tree classifier may be created which is an AI-driven process that is trained to predict which segment (<2 years, 2-5 years, and >5 years) someone is expected to remain in a role or position in based on their capabilities. For example, the decision tree classifier may be analyzed via SHAP library (which uses Shapley values) to determine impacts and uses the BorutaShap library to determine which of the impacts are statistically significant.

Team placement may also be performed using the geometric object fit analysis, as in block 554. Groups of users may also be optimized based on how well they adhere to roles generated by machine learning algorithms. A list of all members of the group may be displayed, as well as their geometric object and the role that has been chosen for them. With an existing group selected, a new “exploratory group” may be created to allow users to experiment with different group compositions to try to improve overall group dynamics according to a role assignment algorithm.

In one example, group optimization may be computed using a clustering algorithm on the grouped candidates (based on their capability values) in order to find common roles between the candidates in a group. The system may then verify which types of roles appear a significant number of times in high performing baseline groups and with what frequency the roles appear. That information can be sent to a group optimization service or component, which solves for a role assignment for every role in the group in order to maximize a match percentage (e.g., 20%, or 90%) for each individual member of the group. The match percent is computed using the Euclidean distance of the capability values from the role capability values, and that distance is converted into a percent by normalizing it according to the maximum distance possible (given capabilities have values from 3 to 9) and then multiplying by 100. The overall group fit may then computed by taking the average of the match percent values of the group members, and anything above 60% is considered High, while anything below 50% is considered Low.

A bonus for those candidates who exceed the criteria for a role, e.g. has the value 6 for a given capability when the condition is at least 3, or the value 1 if the condition is no more than 5. The maximum % of match on the dashboard may be still 100% (even for those exceeding the provided criteria), but the order of candidates with the same percentage of match may be based on the bonus. To calculate the bonus, the difference between the condition and the actual value may be calculated for each capability, and multiplied by a weight of a capability (if weights have been set or provided). Then the bonus may be added to the calculated match percentage. This last operation may be adding absolute values to percentages or additional percentages to percentages. However, the aim is to rank candidates with the same match value on the dashboard, so this computation can work to help distinguish candidates, where the weights are taken into account for the capabilities.

Employee career path development may also be performed by providing geometric modeling of unique development and training paths for current or future employees based on the individual's strengths and weaknesses, as in block 556.

In one example, comparison or filtration may take place using profiles from top performers. The 50 best sales people and their capability profiles may be selected. Their profiles may then be aggregated and averages may be created for statistically relevant capabilities. For instance, a collective average may be 7 in the capability of “drive”, even though some top performers have 8s, 9s, 10s. A new candidate may meet everything else in the ideal geometric shape but the new candidate may also be a 9 in drive. Thus, the new candidate is a good hiring prospect. The aggregation of the top performers can create an ideal candidate in statistically significant capabilities. The comparison instrument can remove statically insignificant traits and then build the geometric object or simply ignore insignificant traits in the comparison process.

Some areas of a person's capabilities may be more relevant to one work profile and less relevant to other work profiles. Candidate capabilities that are relevant to a current comparison for a work profile may be weighted more heavily than other capabilities. Further, a determination on whether or not to recommend the candidate profile can also be based on an aggregation of the amount of deviation measured in weighted capability areas.

In one configuration, the qualitative scoring service 350 (FIG. 3) can score qualitative answers using machine learning that includes deep neural networks. The deep neural network may have been trained by many training examples that are accurate but the answers are subjective or qualitative answers to a question. The deep neural network can be used to determine if the response patterns or written answers provided by a candidate fit the patterns the deep neural network was trained on. If a response is written (or transcribed from audio or video) that does not match correct patterns, then the response may be graded with a lower score or as a less appropriate response. Answers or responses may be processed or tested using a machine learning model that is an LSTM (Long Short Term Memory) type RNN (Recurrent Neural Network). A GPT-2 or BERT machine learning model or related types of models may also be used.

As discussed earlier, the qualitative scoring service 350 can score the qualitative answers by sending the answer to a person in the simulation for scoring. For example, the scoring may be performed by a peer who was also in the same work simulation and has viewed the same business scenario. Alternatively, the scoring could also be performed by a peer who has previously solved the same problems in the same simulation.

This technology can use the geometric object to represent what a high performing employee or individual's capabilities look like. The capabilities that represent the statistically significant differences are identified because they matter to the outcome. Some capabilities are directly correlated to success in the specific job or role, while other capabilities might not statistically correlate to success in a job or role (but might in another job). So, in the geometric shape some capabilities are not relevant because those capabilities do not differ enough from the average to be statically significant. Some capabilities will differ in a positive way and some will differ in a negative way. For example, the correlation may state that a low amount of capability A is seen in top performers, and a high amount of capability B in top performers. This can be represented and compared using the geometric objects. The ideal geometric object may embody the capabilities people can be high or low in for a job or role. When the comparison has been completed, the person can get an aggregate score 83%, 77%, etc. Further, the probability that is calculated for a person as compared to role may be converted to just a text label showing a client a “high” “medium” or “low” probability of producing an outcome the client or administrator has set the system to achieve, as in example. Other labels may be used to represent probabilities as desired. Similarly, a probability of high performance or an attrition likelihood can be calculated using a similar method.

The user may also be presented with their own dashboard representing their own career match benchmarks. For example, the user's or candidate's capabilities may be presented as either exceeding or not exceeding fixed requirements for a role in each capability area. For capabilities, the candidate or user may see their current match score across a single capability dimension in each capability category for which they are not already within a 20% match for not significant capabilities and not within a 10% match for statistically significant capabilities for a specific work profile.

In a more specific example, the match process may first isolate comparable data to enable manual or automatic identification of a desirable candidate. A manually selected desirable candidate can be selected where a client or administrator may select the work profile of an already existing person using a name and/or role of the manually selected ideal candidate. For instance, if John Smith is known to have a strong profile for a certain role, his profile may be used to find other good candidates for the same or similar role.

To reiterate and summarize, automatic selection of an ideal candidate may use averaging of good candidates and good employee samples in a data store. Once the appropriate sample size is reached, statistically significant divergences in the incoming profile from the average baseline profile is identified. Then, an average may be derived from each behavior and capability of the incoming sample profile, which is converted into a geometric map (e.g., 14 and 42 point) that becomes the ideal profile. Each new tested candidate or user's results are similarly converted into a geometric map and the delta (positive, negative or zero) between the two geometric objects is aggregated to provide a match percentage with special weight given to the areas of the map that correspond to statistically significant divergences. This may mean special weight is given to areas where the candidate is very strong compared to the baseline or very weak. The client can also look at candidates beyond their organization and see candidates who are also applicants or interested contractors.

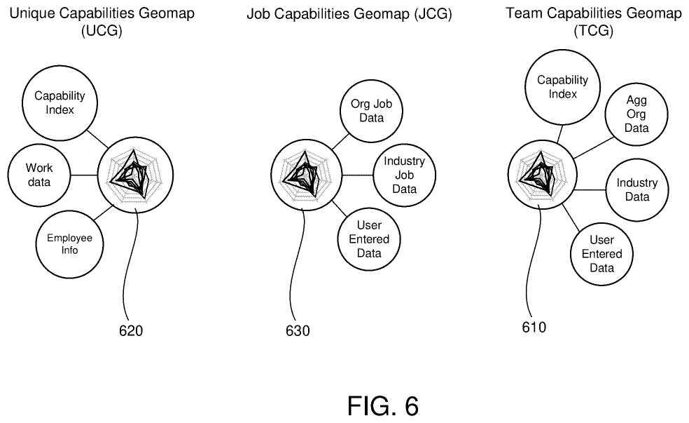

FIG. 6 illustrates that different types of comparisons or matching may be possible, include team matching 610. However, team matching does not aggregate individual profiles together into one profile, as described earlier. Rather, it takes the most similar candidate mapping from each group of candidates for a specific position and aggregates those candidates into position 1, 2, . . . N of a team capabilities geomap (TCG). Then, the matching obtains a geometric shape linked to a separate candidate for each of the N positions. New users are then considered matches to the most similar geometric shape for a position where they have the smallest delta, and that is their match percentage. The technology then identifies the most similar user for each position of all the users that have been sampled. And then identifies the second-best set of users and so on. Then, the total match percentage for the entire user set is provided as the average match. Thus, a prospective team may be identified as a match for a team profile, or a group of individuals may be matched together to build one team. Other information that may be used in addition to the insight index may be: aggregated organizational data, industry data and user entered data (e.g., user entered desired capabilities). The end user can also look at candidates beyond their organization and see candidates who are also applicants or contractors who may be interested in a work profile or position. This may also be filtered by the department.

The technology may be used to identify areas where individuals or groups have unique capabilities 620 (e.g., a capability index) or a unique capabilities geomap (UCG). The benchmark process first isolates comparable data to industry/role subsets. Once the sample size is sufficiently large, this is converted into a geometric map and statistically significant divergence in the sample from the average baseline of all users is identified. Then, an average is derived from each behavior and capability, which is converted into a geometric map (e.g., 14 and 42 point) that becomes the ideal user. Each new tested user's results are similarly converted into a geometric map and the delta (positive, negative or zero) between the two images is aggregated to provide a match percentage with special weight given to the areas of the map that correspond to statistically significant divergences for the individual or the group. Other types of data that can be used to identify unique capabilities may be work data and additional employee data.

A benchmark dashboard may allow a client to see how the roles in their organization map to their industry, how a department maps to their organization (or similar industry departments) and a role compares to their organization average (or similar industry roles).

A job capabilities geomap (JCG) 630 may also be created that represents the desired job capabilities and their desired capability scores for a job or role. Organization job data, industry job data or user entered data may be used for creating the job capabilities geomap (JGC).

A talent marketplace or real time job mapping may also be provided. Due to the ability to be integrated into a client's requisition system, each job posting can provide skills, capabilities, tasks and/or other requirements for each posted role. The client can select any or all internal employees, contractors/temporary and candidates for comparison to the role. The client may select which requirements must have been already collected from candidate in order for a comparison to occur. For each user who has a completed profile, the client can see candidates who match each requisition. Users or candidates may see a match percentage in their profile dashboard to open requisitions and the matches may be ranked by match percentage, which will link to the posted job descriptions.

If their candidate profile is complete, the candidate can press a single button to submit their full profile, resume and/or application. The candidate may be prompted to include a cover letter, which may be added to the submission or declined if the candidate prefers. If their profile is not complete, candidates or users may be prompted to complete their profile by populating the incomplete information—skills, task experience, resume and type of work they want (FullTime (FT), PartTime (PT), Gig, Contract). In this system, clients can search solely for skills, job capabilities or tasks enabling them to access the gig economy and contract economy workers with ease. Clients may even hire candidates for a single project.

The technology may provide a talent marketplace matching process on the client side (e.g., for a company). A client requisition system would have or accept an add-on that enables data to be stored on all the following requirements: FullTime/PartTime/Contract/Short Term, internal/candidate/contract/temp, education, experience, skills, tasks and capabilities requirements. A requirement can be set to null and not be needed. When a position is filled, the system would close the position. Until closed, this data about work profile requirements may flow in a data store in real time. Currently available candidates may be searched for as soon as the requisition is opened via a talent scan system and as filtered by the requisition. The client user would see only candidates who met the objective requirements as ordered by match % using a talent scan algorithm.

The talent marketplace can have a matching process on the user side (e.g., the candidate/employee side). A user can complete their work profile measurements and access their profile. Beneath their work profile, a section may be provided for the user which displays currently available, high match jobs to open requisitions for which the user appears to be a good match. This feature can be enabled, disabled, or show only positions available at a specific client organization.

In order for this searching feature to be activated, this section may initially prompt the user to complete further information. The candidate or user can indicate whether they are not looking for work or looking for FT/PT/Contract/Short Term. The candidate may indicate their role(s) desired, and then take the skill survey/measurements associated with those roles. The candidate or user can populate tasks they believe they are proficient in doing (e.g., working with a Fulcrum database). The user may also indicate their level of education and years of experience. After receiving this information, available roles can automatically populate for the candidate or user whenever they log into their user profile and update with live results each time the candidate or user logs in. When submitting for their first role, the user may be prompted to upload their resume. If they do, the resume can be automatically submitted to the client requisition system, with a prompt each time regarding whether they want to include a cover letter. If the candidate or user does not submit a cover letter, they are linked to the client requisition posting. Thus, the live data from clients can be used to direct users (using their personal profile) toward positions that are high probability matches for the user in real time

This technology may be customized to other vertical industry segments to make the system work better in specific contexts. For example, the capabilities can be made more specific for vertical industries such as food industry or manufacturing. This way the comparision instrument may be modified and managed to have input feeds and perform comparisons that represent capabilities tailored to the industry vertical. For example, the simulation tests may be customized to manufacturing problems (e.g., simulated manufacturing tests are given), etc. The comparison instrument can perform analysis on the candidate, simulator data, and employer inputs and can be used for different applications.

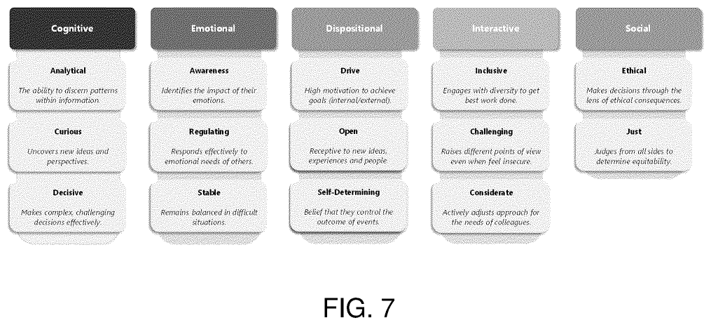

This technology is also an iterative learning system. When new employees are hired by an employer, their profiles are stored into the system and these new profiles (along with on-going performance evaluations) can improve the system. The candidate profiles, employee profiles, and simulation feed streams can train the machine over time and this changes the way the comparison instrument or learning machine behaves. The new employees change the matching probabilities for the next candidate and the probabilities for attrition also change over time. In addition, data stored in the system or client HCM can also be shared data (e.g. human profiles vs objective HCM outcomes such as tenure, promotions, etc.) and this shared data can be used to feed the learning system. The system is constantly creating additional connections for causality, potential causality or linkages. Since the input feed stream is candidate data or profiles of people that engage in the work simulation application, a comparison can be made to existing data that is internal company data or industry norms. The streams are the candidate information but based on the hiring outcomes there are ongoing changes to the baseline for the company data, industry aggregation data or globally aggregated data. FIG. 7 illustrates a list of employee or candidate capabilities. These capabilities may be divided into categories such as: cognitive, emotional, dispositional, interactive and social. For example, under the cognitive category there may be capabilities such as analytical, curious, and decisive that may be measured by the work simulation application.

An organization that has completed a sufficient sample size for a role or work profile will have data that can be imported into their career mapping system that includes each statistically significant capability and other requirements (e.g. years of experience, education, skills) for the role. An employee can navigate the career mapping system via their profile by looking at ranked roles based on a % match or searching for specific roles they want. By clicking on the role, they will see a comparison of their current profile with the recommended profile of the role.

Each skill, capability and requirement may be manually mapped to client's L&D (Learning and Development) coursework system. In the role profile, they may receive specific recommendations based on the skill or capability where they do not yet meet that role's requirements, and this may enable every user to tailor their coursework through a provided LMS (Learning Management System).

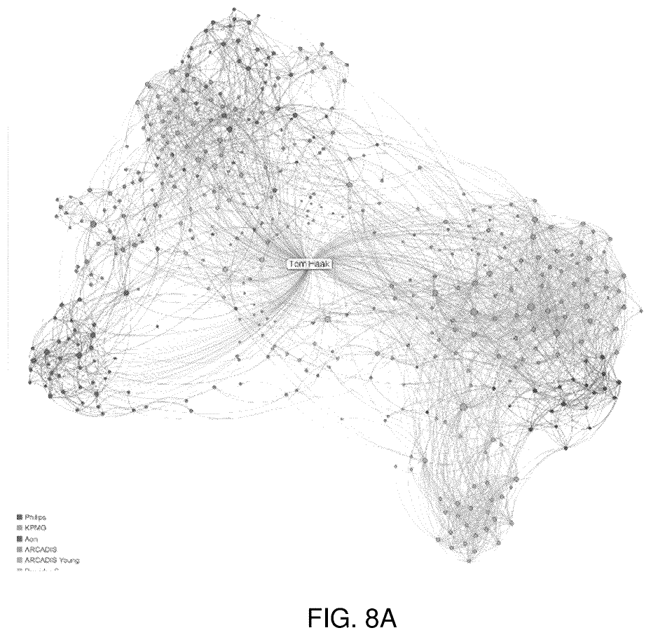

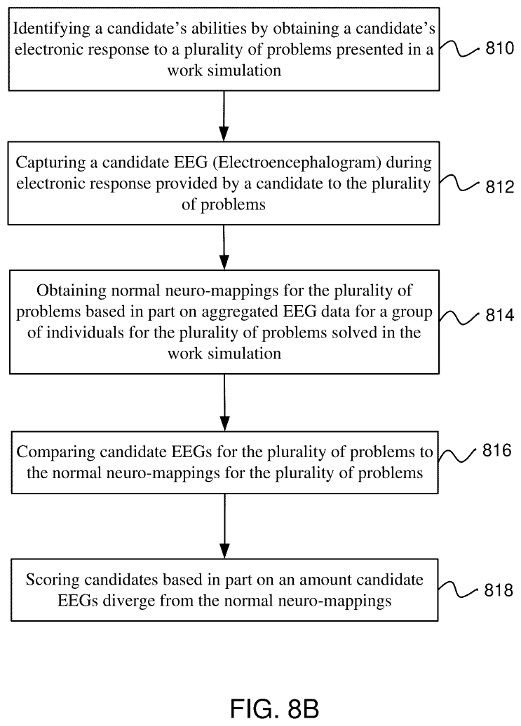

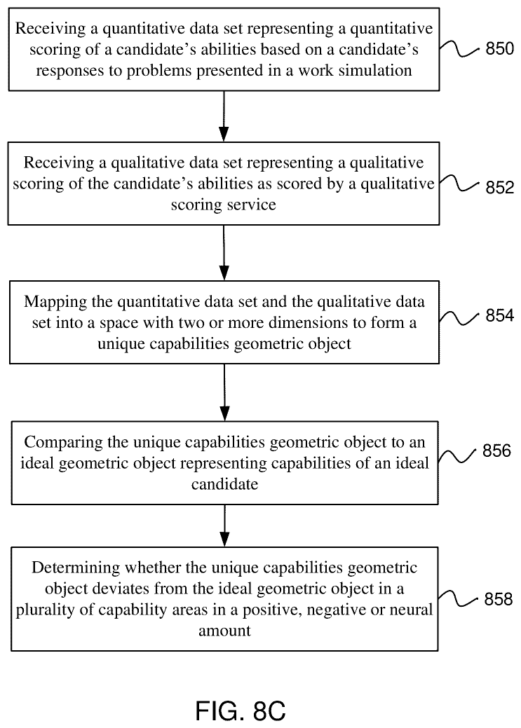

FIG. 8A illustrates an example of an influencer and social networks identified within an organization. Organization analysis may be used in the construction of change champion networks to facilitate organization transformation. By utilizing data from employee communication systems (e-mail, teams/skype, etc.) organizations can be visualized as having silos, connectors, and influencers. Of the connectors and influencers, their openness, drive and other related capabilities are compared to identify those individuals most likely to effectively support change. The client then receives a match ranked list showing a degree of influence and change orientation for individuals.

The system can also identify organization influencer scores. This sub-system can aggregate a number of communications against department benchmarks. In an example, the average number of e-mails per day is 20 and 30 internal Skype communications in the finance group. A user may then be identified as being 1, 2 or 3 standard deviations (SD) above or below this baseline. The system can also compare communications between departments. More specifically, certain users may communicate across departments or silos in the company. For example, a user in department X may communicate much more often with department Y than other users. Thus, the user is a connector between these two silos. In this more specific example, the average number of e-mails to non-finance users per day is 3 and 10 internal Skype communications. Based on this example, a user can then be defined as being 1, 2 or 3 standard deviations above or below this baseline. These scorings can produce an influence score for the former and connector score for the latter. A change champion dashboard can show an organization network map highlighting influencers above SD 2 and connectors above SD 2 who also have 80% (using talent scan algorithm) match capabilities in openness, curious, challenging and drive behaviors. FIG. 8 illustrates an example graph of an influencer in an organization.

This technology provides a fast, engaging approach for rapidly measuring more than a dozen high demand human capabilities. The number of individuals who use the system can be scaled up quickly, depending on the amount of work simulation hardware available. Users can step into a contextual, digital work sample that uses a variety of multi-media experiences such as game-based simulations, video questions and cognitive assessments to enable an employer learn about the user.

FIG. 8B illustrates a flowchart for a method for determining a candidate's suitability for a work profile. The method may include identifying a candidate's abilities by obtaining a candidate's electronic response to a plurality of problems presented in a work simulation, as in block 810. The candidate's abilities may be scored by a work simulator or work simulation application, and the candidate's abilities may be represented in a capability index.

A candidate EEG (Electroencephalogram) may be captured during electronic response provided by a candidate to the plurality of problems, as in block 812. Normal neuro-mappings may be obtained or identified for the plurality of problems based in part on aggregated EEG data for a group of individuals for the plurality of problems solved in the work simulation, as in block 814.

Candidate EEGs for the plurality of problems may be compared to the normal neuro-mappings for the plurality of problems, as in block 816. The candidates may be scored based in part on an amount candidate EEGs diverge from the normal neuro-mappings, as in block 818.

The scoring may be adjusted based on dishonesty or uncertainty detected by the work simulation. For example, the raw scoring of answers or feedback from the work simulation may be adjusted based on detected dishonesty or uncertainty as determined using the candidate EEGs as compared to normal neuro-mappings. The scoring may also be adjusted based on the candidate answering questions with emotional material to identify sociopathy or psychopathy.

Facial expression recognition may be used to detect incorrect emotional or mental states. For example, a facial expression may be mapped to determine whether the candidate has neurodivergent behavior or the candidate is lying or confused.

FIG. 8C is a flowchart illustrating an example of a method for comparing a candidate's capability profile to a work profile. The method may include receiving a quantitative data set representing a quantitative scoring of a candidate's abilities based on a candidate's responses to problems presented in a work simulation, as in block 850. Another operation may be receiving a qualitative data set representing a qualitative scoring of the candidate's abilities as scored by a qualitative scoring service, as in block 852. The capabilities of a candidate that are not statistically significant to success in the work profile can be filtered out, and the filtering out of capabilities may take place before or after the unique capabilities geometric object is created. The qualitative scoring service may score qualitative answers using machine learning that includes deep neural networks. In contrast, the qualitative scoring service may score qualitative answers by sending an answer to a person or peer for scoring.

The quantitative data set and the qualitative data set may be mapped into a space with two or more dimensions to form a unique capabilities geometric object, as in block 854.

The unique capabilities geometric object may be compared to an ideal geometric object representing capabilities of an ideal candidate, as in block 856. A determination may be made regarding whether the unique capabilities geometric object deviates from the ideal geometric object in a plurality of capability areas in a positive, negative or neural amount. This allows a determinization to be made regarding whether or not to recommend the candidate's capability profile based on an aggregation of an amount of deviation measured in capability areas. Further, the technology may determine whether or not to recommend the candidate's capability profile based on an aggregation of an amount of deviation measured using weighted capability areas.

The comparison may also be made based an area or volume of an object that represents the candidate's capabilities as compared to an area or volume an object representing the desired candidate capabilities or ideal capabilities. For example, a 2D or 3D unique capabilities geometric object can be compared to a 2D or 3D ideal geometric object representing capabilities of an ideal candidate by comparing areas or volumes.

The comparison processes may also use a decision tree classifier to determine whether qualitative attributes of the candidate fit a role. In another comparison example, the unique capabilities geometric object can be compared to an ideal geometric object by computing a match percentage using the Euclidean distance of the capability values from the role capability values. The Euclidian distance may be converted into a percent by normalizing the distance according to a maximum distance possible, using capabilities values from 3 to 9 or 1 to 10, and then multiplying by 100.

In another determination process, an attrition value can be determined using a decision tree classifier to predict an attrition segment for a candidate based on the candidate's capabilities.

Another comparison process may apply clustering on grouped candidates to find common roles of candidates in the group. The types of roles that appear a significant number of times in high performing baseline groups and with what frequency the roles appear may be determined. A new group with roles that match the types of roles and the frequency the roles appear in high performing groups may then be formed.

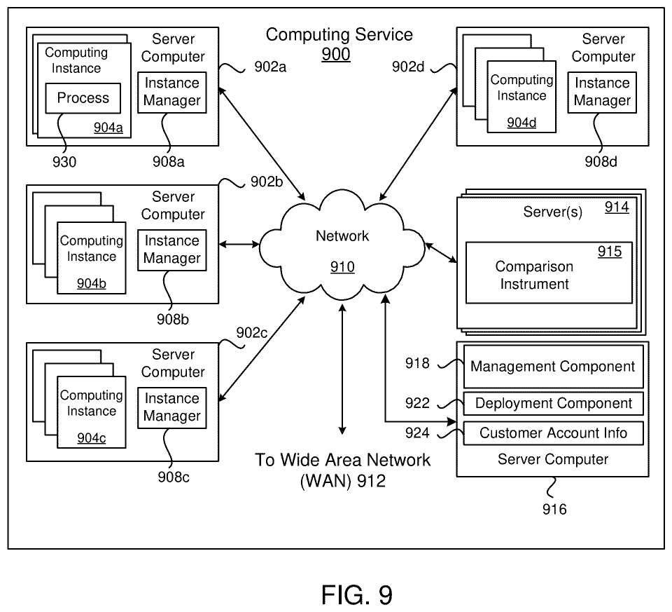

FIG. 9 is a block diagram illustrating an example computing service 900 that may be used to execute and manage a number of computing instances 904a-d upon which the present technology may execute. In particular, the computing service 900 depicted illustrates one environment in which the technology described herein may be used. The computing service 900 may be one type of environment that includes various virtualized service resources that may be used, for instance, to host computing instances 904a-d that execute processes 930.

The computing service 900 may be capable of delivery of computing, storage and networking capacity as a software service to a community of end recipients. In one example, the computing service 900 may be established for an organization by or on behalf of the organization. That is, the computing service 900 may offer a “private cloud environment.” In another example, the computing service 900 may support a multi-tenant environment, wherein a plurality of customers may operate independently (i.e., a public cloud environment). Generally speaking, the computing service 900 may provide the following models: Infrastructure as a Service (“IaaS”) and/or Software as a Service (“SaaS”). Other models may be provided. For the IaaS model, the computing service 900 may offer computers as physical or virtual machines and other resources. The virtual machines may be run as guests by a hypervisor, as described further below.

Application developers may develop and run their software solutions on the computing service system without incurring the cost of buying and managing the underlying hardware and software. The SaaS model allows installation and operation of application software in the computing service 900. End customers may access the computing service 900 using networked client devices, such as desktop computers, laptops, tablets, smartphones, etc. running web browsers or other lightweight client applications, for example. Those familiar with the art will recognize that the computing service 900 may be described as a “cloud” environment.

The particularly illustrated computing service may include a plurality of server computers 902a-d. The server computers 902a-d may also be known as physical hosts. While four server computers are shown, any number may be used, and large data centers may include thousands of server computers. The computing service 900 may provide computing resources for executing computing instances 904a-d. Computing instances 904a-d may, for example, be virtual machines. A virtual machine may be an instance of a software implementation of a machine (i.e., a computer) that executes applications like a physical machine. In the example of a virtual machine, each of the server computers 902a-d may be configured to execute an instance manager 908a-d capable of executing the instances. The instance manager 908a-d may be a hypervisor, virtual machine manager (VMM), or another type of program configured to enable the execution of multiple computing instances 904a-d on a single server. Additionally, each of the computing instances 904a-d may be configured to execute one or more applications.

A server 914 may be reserved to execute software components for implementing the present technology. For example, the server 914 or computing instance may include a comparison instrument 915 for comparing capability profiles and/or one or more machine learning models to process qualitative answers received.

A server computer 916 may execute a management component 918. A customer may access the management component 918 to configure various aspects of the operation of the computing instances 904a-d purchased by a customer. For example, the customer may setup computing instances 904a-d and make changes to the configuration of the computing instances 904a-d.

A deployment component 922 may be used to assist customers in the deployment of computing instances 904a-d. The deployment component 922 may have access to account information associated with the computing instances 904a-d, such as the name of an owner of the account, credit card information, country of the owner, etc. The deployment component 922 may receive a configuration from a customer that includes data describing how computing instances 904a-d may be configured. For example, the configuration may include an operating system, provide one or more applications to be installed in computing instances 904a-d, provide scripts and/or other types of code to be executed for configuring computing instances 904a-d, provide cache logic specifying how an application cache is to be prepared, and other types of information. The deployment component 922 may utilize the customer-provided configuration and cache logic to configure, prime, and launch computing instances 904a-d. The configuration, cache logic, and other information may be specified by a customer accessing the management component 918 or by providing this information directly to the deployment component 922.

Customer account information 924 may include any desired information associated with a customer of the multi-tenant environment. For example, the customer account information may include a unique identifier for a customer, a customer address, billing information, licensing information, customization parameters for launching instances, scheduling information, etc. As described above, the customer account information 924 may also include security information used in encryption of asynchronous responses to API requests. By “asynchronous” it is meant that the API response may be made at any time after the initial request and with a different network connection.

A network 910 may be utilized to interconnect the computing service 900 and the server computers 902a-d, 916. The network 910 may be a local area network (LAN) and may be connected to a Wide Area Network (WAN) 912 or the Internet, so that end customers may access the computing service 900. In addition, the network 910 may include a virtual network overlaid on the physical network to provide communications between the servers 902a-d. The network topology illustrated in FIG. 4 has been simplified, as many more networks and networking devices may be utilized to interconnect the various computing systems disclosed herein.

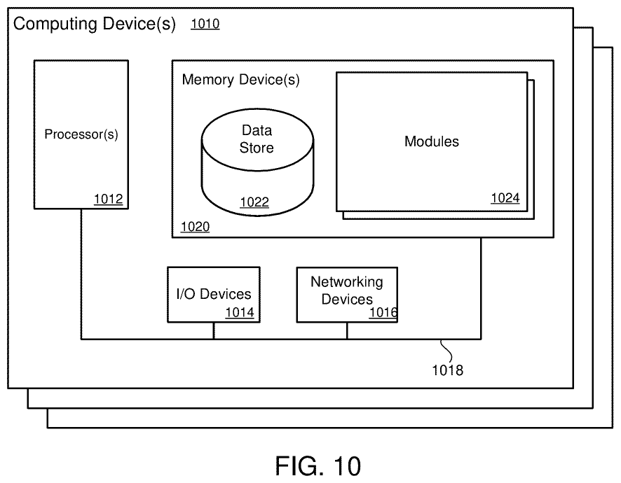

FIG. 10 illustrates a computing device 1010 which may execute the foregoing subsystems of this technology. The computing device 1010 and the components of the computing device 1010 described herein may correspond to the servers and/or client devices described above. The computing device 1010 is illustrated on which a high-level example of the technology may be executed. The computing device 1010 may include one or more processors 1012 that are in communication with memory devices 1020. The computing device may include a local communication interface 1018 for the components in the computing device. For example, the local communication interface may be a local data bus and/or any related address or control busses as may be desired.

The memory device 1020 may contain modules 1024 that are executable by the processor(s) 1012 and data for the modules 1024. For example, the memory device 1020 may include an inflight interactive system module, an offerings subsystem module, a passenger profile subsystem module, and other modules. The modules 1024 may execute the functions described earlier. A data store 1022 may also be located in the memory device 1020 for storing data related to the modules 1024 and other applications along with an operating system that is executable by the processor(s) 1012.

Other applications may also be stored in the memory device 1020 and may be executable by the processor(s) 1012. Components or modules discussed in this description that may be implemented in the form of software using high programming level languages that are compiled, interpreted or executed using a hybrid of the methods.

The computing device may also have access to I/O (input/output) devices 1014 that are usable by the computing devices. An example of an I/O device is a display screen that is available to display output from the computing devices. Other known I/O device may be used with the computing device as desired. Networking devices 1016 and similar communication devices may be included in the computing device. The networking devices 1016 may be wired or wireless networking devices that connect to the internet, a LAN, WAN, or other computing network.

The components or modules that are shown as being stored in the memory device 1020 may be executed by the processor 1012. The term “executable” may mean a program file that is in a form that may be executed by a processor 1012. For example, a program in a higher-level language may be compiled into machine code in a format that may be loaded into a random-access portion of the memory device 1020 and executed by the processor 1012, or source code may be loaded by another executable program and interpreted to generate instructions in a random-access portion of the memory to be executed by a processor. The executable program may be stored in any portion or component of the memory device 1020. For example, the memory device 1020 may be random access memory (RAM), read only memory (ROM), flash memory, a solid-state drive, memory card, a hard drive, optical disk, floppy disk, magnetic tape, or any other memory components.

The processor 1012 may represent multiple processors and the memory 1020 may represent multiple memory units that operate in parallel to the processing circuits. This may provide parallel processing channels for the processes and data in the system. The local interface 1018 may be used as a network to facilitate communication between any of the multiple processors and multiple memories. The local interface 1018 may use additional systems designed for coordinating communication such as load balancing, bulk data transfer, and similar systems.

While the flowcharts presented for this technology may imply a specific order of execution, the order of execution may differ from what is illustrated. For example, the order of two more blocks may be rearranged relative to the order shown. Further, two or more blocks shown in succession may be executed in parallel or with partial parallelization. In some configurations, one or more blocks shown in the flow chart may be omitted or skipped. Any number of counters, state variables, warning semaphores, or messages might be added to the logical flow for purposes of enhanced utility, accounting, performance, measurement, troubleshooting or for similar reasons.

Some of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of executable code may, for instance, comprise one or more blocks of computer instructions, which may be organized as an object, procedure, or function. Nevertheless, the executables of an identified module need not be physically located together, but may comprise disparate instructions stored in different locations which comprise the module and achieve the stated purpose for the module when joined logically together.

Indeed, a module of executable code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices. The modules may be passive or active, including agents operable to perform desired functions.

The technology described here can also be stored on a computer readable storage medium that includes volatile and non-volatile, removable and non-removable media implemented with any technology for the storage of information such as computer readable instructions, data structures, program modules, or other data. Computer readable storage media include, but is not limited to, RAM, ROM, EEPROM, flash memory or other memory technology, CD-ROM, digital versatile disks (DVD) or other optical storage, magnetic cassettes, magnetic tapes, magnetic disk storage or other magnetic storage devices, or any other computer storage medium which can be used to store the desired information and described technology.

The devices described herein may also contain communication connections or networking apparatus and networking connections that allow the devices to communicate with other devices. Communication connections are an example of communication media. Communication media typically embodies computer readable instructions, data structures, program modules and other data in a modulated data signal such as a carrier wave or other transport mechanism and includes any information delivery media. A “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency, infrared, and other wireless media. The term computer readable media as used herein includes communication media.

Furthermore, the described features, structures, or characteristics may be combined in any suitable manner in one or more examples. In the preceding description, numerous specific details were provided, such as examples of various configurations to provide a thorough understanding of examples of the described technology. One skilled in the relevant art will recognize, however, that the technology can be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements can be devised without departing from the spirit and scope of the described technology.

1. A method for determining a candidate's suitability for a work profile, comprising:
identifying a candidate's abilities by obtaining a candidate's electronic response to a plurality of problems presented in a work simulation;
capturing a candidate EEG (Electroencephalogram) during electronic response provided by a candidate to the plurality of problems;
obtaining normal neuro-mappings for the plurality of problems based in part on aggregated EEG data for a group of individuals for the plurality of problems solved in the work simulation;
comparing candidate EEGs for the plurality of problems to the normal neuro-mappings for the plurality of problems; and
scoring candidates based in part on an amount candidate EEGs diverge from the normal neuro-mappings.

2. The method of claim 1, further comprising adjusting the scoring based on dishonesty or uncertainty detected by the work simulation.

3. The method of claim 1, further comprising adjusting raw scoring of answers or feedback from the work simulation based on detected dishonesty or uncertainty as determined using the candidate EEGs as compared to normal neuro-mappings.

4. The method of claim 1, further adjusting the scoring based on the candidate answering questions with emotional material to identify sociopathy or psychopathy.

5. The method as in claim 1, further comprising using facial expression recognition to detect incorrect emotional or mental states.

6. The method as in claim 1, further comprising mapping a facial expression to determine whether the candidate has neurodivergent behavior or the candidate is lying or confused.

7. A method for comparing a candidate's capability profile to a work profile, comprising:
receiving a quantitative data set representing a quantitative scoring of a candidate's abilities based on a candidate's responses to problems presented in a work simulation;
receiving a qualitative data set representing a qualitative scoring of the candidate's abilities as scored by a qualitative scoring service;
mapping the quantitative data set and the qualitative data set into a space with two or more dimensions to form a unique capabilities geometric object;
comparing the unique capabilities geometric object to an ideal geometric object representing capabilities of an ideal candidate; and
determining whether the unique capabilities geometric object deviates from the ideal geometric object in a plurality of capability areas in a positive, negative or neural amount.

8. The method as in claim 7, further comprising filtering out capabilities of a candidate that are not statistically significant to success in the work profile, wherein the filtering out capabilities takes place before or after the unique capabilities geometric object is created.

9. The method as in claim 7, determining whether or not to recommend the candidate's capability profile based on an aggregation of an amount of deviation measured in capability areas.

10. The method as in claim 7, determining whether or not to recommend the candidate's capability profile based on an aggregation of an amount of deviation measured using weighted capability areas.

11. The method as in claim 7, wherein the qualitative scoring service scores qualitative answers using machine learning that includes deep neural networks.

12. The method as in claim 11, wherein the qualitative scoring service scores qualitative answers by sending an answer to a person for scoring.

13. The method as in claim 11, wherein the qualitative scoring service scores qualitative answers by sending an answer to a peer in the work simulation for scoring.

14. The method as in claim 7, further comprising sending quantitative answers to the quantitative scoring service for scoring and entry in the candidate's capability profile.

15. The method as in claim 7, further comprising comparing the unique capabilities geometric object to an ideal geometric object representing capabilities of an ideal candidate by comparing areas or volumes.

16. The method as in claim 7, further comprising using a decision tree classifier to determine whether qualitative attributes of the candidate fit a role.

17. The method as in claim 7, further comprising comparing the unique capabilities geometric object to an ideal geometric object by computing a match percentage using a Euclidean distance of capability values from role capability values.

18. The method as in claim 17, wherein the Euclidian distance is converted into a percent by normalizing the Euclidian distance according to a maximum distance possible, using capabilities values from 1 to 10, and multiplying by 100.

19. The method as in claim 7, further comprising determining an attrition value using a decision tree classifier to predict an attrition segment for a candidate based on the candidate's capabilities.

20. The method as in claim 7, further comprising:
applying clustering on grouped candidates to find common roles of candidates in the group;
determining types of roles that appear a significant number of times in high performing baseline groups and with what frequency the roles appear; and
forming a new group with roles that match the types of roles and the frequency the roles appear in high performing groups.
